(19) United States
(12) Patent Application Publication
(10) Pub. No.: US 2025/0228809 A1
(43) Pub. Date: Jul. 17, 2025

(54) COMPOSITIONS FOR USE

(71) Applicant: SOCIETE DES PRODUITS NESTLE S.A., Vevey (CH)

(72) Inventors: SAGAR THAKKAR, La Tour-de-Peilz (CH); TINU MARY SAMUEL, La Tour-de-Peilz (CH); FRANCESCA GIUFFRIDA, Mezieres (CH)

(21) Appl. No.: 18/695,440

(22) PCT Filed: Sep. 26, 2022
(86) PCT No.: PCT/EP2022/076668

§ 371 Date: Mar. 26, 2024

(30) Foreign Application Priority Data
Sep. 28, 2021 (EP) 21199525.3

(51) Int. Cl. A61K 31/202; A23L 33/00; A61K 31/07; A61K 31/4415; A61K 31/51; A61K 31/519; A61K 31/525; A61K 31/675; A61K 31/702; A61K 31/7032; A61K 33/06; A61K 33/42

(52) U.S. Cl. CPC A61K 31/202; A23L 33/40; A61K 31/07; A61K 31/4415; A61K 31/51; A61K 31/519; A61K 31/525; A61K 31/675; A61K 31/702; A61K 31/7032; A61K 33/06; A61K 33/42

(57) ABSTRACT

A paediatric supplement or human milk fortifier composition comprising at least one nutrients selected in the list consisting of: ALA, DHA, EPA, 2FL, LNnT, vitamin A, thiamine, thiamine monophosphate, vitamin B2, vitamin B6, vitamin B9, calcium and phosphorus, GD3, GM3. Said paediatric supplement or human milk fortifier composition is tailored or adapted for a child born via C-section who is breast fed and/or to fortify the breast milk of a woman who has given birth via C-section. Said paediatric supplement or human milk fortifier composition is beneficial to provide adequate nutrition to a child born via C-section and promote associated health benefits.

### TECHNICAL FIELD OF THE INVENTION

The present invention relates to a supplement or to a human milk fortifier composition. In particular, the present invention relates to a supplement and/or a human milk fortifier composition specifically tailored for a child born via caesarean section. The invention furthermore relates to the use of said supplement or milk fortifier composition.

### BACKGROUND OF THE INVENTION

Caesarean section rates are increasing around the world; whilst the international health care community considers that an ideal caesarean section rate to be between 10 to 15%, it is reported that today's rate may be higher than 30% in many countries (Boerma T. et al., Lancet: 2018: 392 (10155): 1341-1348).

The reasons for caesarean sections vary. In many cases a caesarean section is medically indicated and therefore necessary to save the life of the baby and/or the mother, this may be because a vaginal birth is not possible, e.g. because of failure of the normal progression of labor. In other cases, a caesarean section may be elective and performed at the request of a pregnant women or family.

Regardless of the reason for a caesarean section, children born in this way are reported to be at an increased risk of suffering from a variety of health issues in infancy, childhood, and even later in life. The reasons for this increased risk are not clear. However, given the increasing frequency of caesarean sections, there is a need to identify factors that may contribute to this risk and to address them.

The inventors have identified a factor that may contribute to this risk. In particular the inventors have found that the concentration of several nutrients found in human breast milk (hereinafter “HM”) produced by mothers to children born via caesarean section (hereinafter C-section) may differ from the concentration of the same nutrients found in HM produced by mothers to children born via vaginal delivery. More particularly the inventors have found that the concentration of certain specific Human Milk Oligosaccharides (hereinafter “HMOs”), vitamins, fatty acids and minerals found in HM produced by mothers to children born via vaginal delivery may be higher than the concentration of the same specific HMOs, vitamins, fatty acids and minerals found in HM produced by mothers to children born via C-section delivery.

Accordingly, there is need for paediatric supplements or milk fortifiers comprising one or more nutrients that can be used to fortify HM produced by mothers who have given birth by C-section, and to optimise the intake of one or more nutrients, such as HMOs, vitamins, fatty acids and minerals, in children delivered via C-section.

### SUMMARY OF THE INVENTION

The inventors have surprisingly found that the concentration of certain specific nutrients in HM produced by mothers to children born by vaginal delivery may be higher than the concentration of the same nutrients found in HM produced by mothers to children born by C-section. An optimal intake of these compounds in infancy is believed to be necessary to ensure optimum health and development. Providing one or more nutrients that were observed to be decreased in HM of woman who has delivered via C-section to a child born via C-section may allow restoring the levels of those nutrients to a level at least comparable to the concentrations of the same nutrients found in HM produced by mother to a child born via vaginal delivery. Considering these findings, the inventors have developed a paediatric supplement or human milk fortifier composition comprising at least one or more of such nutrients. Namely, the specific nutrients above mentioned are: alpha-linolenic fatty acid (hereinafter “ALA”), docosahexaenoic acid (hereinafter “DHA”), eicosapentaenoic acid (hereinafter “EPA”), 2-fucosyl lactose (hereinafter “2′FL”), lacto-N-neotetraose (hereinafter “LNnT”), vitamin A, thiamine, thiamine monophosphate, vitamin B2, vitamin B6, vitamin B9, calcium and phosphorus. and gangliosides GD3 and GM3 (herein after “GD3” and “GM3”, respectively).

The invention is set out in the claims and in the detailed description included herein.

In one aspect, the present invention provides for a paediatric supplement or human milk fortifier composition comprising at least one nutrient selected in the group consisting of:
alpha linolenic acid (ALA), for example in a daily amount ranging from 19 to 80 mg;
docosahexaenoic acid (DHA), for example in a daily amount ranging from 13 to 25 mg;
eicosapentanoic acid (EPA), for example in a daily amount ranging from 6 to 9 mg;
2-fucosyl lactose (2′-FL), for example in a daily amount ranging from 280 to 1050 mg;
lacto-N-neotetraose (LNnT), for example in a daily amount ranging from 30 to 50 mg;
vitamin A, for example in a daily amount ranging from 224 to 342 μg;
thiamine, for example in a daily amount ranging from 6.6 to 10 μg;
thiamine monophosphate, for example in a daily amount ranging from 10 to 15 μg;
vitamin B2, for example in a daily amount ranging from 9 to 19 μg;
vitamin B6, for example in a daily amount ranging from 15 to 68 μg;
vitamin B9, for example in a daily amount ranging from 1.1 to 9 μg;
calcium, for example in a daily amount ranging from 10 to 16 mg;
phosphorus, for example in a daily amount ranging from 4.5 to 14 mg;
GD3, for example in a daily amount ranging from 8 to 12 mg; and
GM3, for example in a daily amount ranging from 1 to 1.5 mg;
or any combination thereof.

In another aspect, the present invention also provides for a paediatric supplement or human milk fortifier composition as above described for use in preventing nutrient inadequacy in a child born via C-section.

Children born via C-section, compared with those born vaginally, are at increased risk of altered immune development, allergy, atopy, asthma, and reduced diversity of the intestinal gut microbiome (Sandall J. et al., Lancet: 2018: 392 (10155): 1349-1357). Recent evidence also suggests that C-section may be associated with neurodevelopmental outcomes such as a shift in brain development, at least during early infancy (e.g. significantly lower white matter development in widespread brain regions and significantly lower functional connectivity in the brain default mode network) (Deoni S C. et al., Am. J. Neuroradiol.: 2019: 40 (1): 169-177).

Accordingly, children born via C-section are in general at increased risk of suboptimal growth and development.

In a further aspect, the present invention also provides for a paediatric supplement or human milk fortifier composition as above described for use in preventing sub-optimal growth and development in a child born via C-section. As previously discussed, one factor that may contribute to suboptimal growth and development in a child born via C-section is that the concentration of several nutrients found in HM produced by mothers to children born via C-section may differ from the concentration of the same nutrients found in HM produced by mothers to children born by vaginal delivery. An optimal intake of these nutrients in early childhood (i.e. while the child is still breast fed) is believed to be necessary to ensure optimum health and development. Therefore, providing a paediatric supplement or human milk fortifier composition that delivers one or more nutrients that are decreased in HM produced by mothers to children born via C-section may address the nutrient inadequacy in said children. Consequently, said children born via C-section may intake an optimized amount of nutrients, thereby preventing the risk of sub-optimal growth and development in said children.

As above discussed, certain HMOs have been found by the inventors to be present in decreased amounts in the HM of women who delivered via C-section.

HMOs are, collectively, the third largest solid constituent in HM, and a variety of benefits have been associated with their intake. Consequently, an optimal intake of these compounds in early childhood is believed to be necessary to ensure optimum health and development. HMOs have for example been linked to a variety of biological functions including the establishment of gut microbiota, the composition of which has been identified as differing between children delivered via C-section and children delivered vaginally, e.g. in the first six months of life.

HMOs are responsible for directly stimulating the immune system by promoting good gut bacteria, strengthening the gut barrier function and blocking pathogens (Blanton et al., Science: 2016: 24: 352 (6293): 1533; Arrieta et al., Front Immunol.: 2014: 5: 427; Cheng Y J et al., Pediatr. Neonatol.: 2021: doi.org/10.1016/j.pedneo.2020.12.013). HMOs shape the establishing early life gut microbiota and supposedly help the development of appropriate immune competence. This is particular evident in situations of an early life dysbiotic microbiota such as seen upon C-section birth (Sprenger et al., Eur. J. Nutr. 2017: 56(3):1293-1301; Korpela et al., Sci. Rep. 2018: 8(1):13757). Through their actions on gut and on microbiota, HMOs positively influence the infants' mucosal and systemic immunity, many of these actions are exerted through carbohydrate-carbohydrate interactions with pathogens or host cells. (Donovan S M et al., Ann. Nutr. Metab.: 2017: 69: Suppl. 2: 42-51).

In an additional aspect, the present invention provides for a paediatric supplement or human milk fortifier composition as herein described for use in optimising the gut microbiota and/or preventing sub-optimal gut microbiota in a child born via C-section.

As above discussed, certain omega-3 fatty acids have also been found by the present inventors to be present in decreased amounts in the human breast milk of women who delivered via C-section.

Omega-3 fatty acids ALA, EPA and DHA act as precursor of some inflammation resolving compounds such as prostaglandins, leukotrienes and resolvins (Ishiara T. et al., International Immunology: 2019: 31 (9): 559-567). The observation that ALA and DHA were present at lower levels in HM produced by mothers who have delivered via C-section indicates that ALA and DHA were presumably converted to these protective molecules and have therefore reduced availability for children by HM. Therefore, the intake by a C-section born child of a paediatric supplement or human milk fortifier composition that provides extra ALA, EPA and DHA (omega-3 fatty acids) may address the nutrient inadequacy in said child. Consequently, said child born via C-section may intake an optimized amount of nutrients, thereby preventing the risk of inflammatory process in said child.

In a still further aspect, the present invention provides for a paediatric supplement or human milk fortifier composition as herein described for use in the prevention and/or treatment of inflammatory process in a child born via C-section.

As above discussed, certain omega-3 fatty acids have also been found by the inventors to be present in decreased amounts in the HM of women who delivered via C-section.

Among omega 3 fatty acids, ALA is essential and is the most abundant omega-3 fatty acid in the diet. ALA is the precursor of long-chain omega-3 polyunsaturated fatty acids (PUFAs) including EPA (20:5n-3), docosapentaenoic acid (DPA; 22:5n-3), and DHA (22:6n-3) (Leonarda et al., Progress in Lipid Research: 2004: 43: 36-5). These long-chain omega-3 PUFAs can be also consumed directly from food sources such as oily fish. Animal studies demonstrate that consuming a diet lacking in omega-3 fatty acids during pregnancy and lactation results in neurological abnormalities in offspring, e.g. impairments of cognitive and visual function (Brenna J. T. et al., Maternal and Child Nutrition: 2011: 7 (s2): 59-79; Lauritzen et al., Progress in Lipid Research: 2001: 40: 1-94) and that these impairments are associated with a reduction in brain DHA content. Therefore, the intake by a C-section born child of a paediatric supplement or human milk fortifier composition that provides extra ALA may address the nutrient inadequacy in said child. Consequently, said child born via C-section may intake an optimized amount of nutrients, thereby promoting cognitive development in said child.

In a still further aspect, the present invention provides for paediatric supplement or human milk fortifier composition as herein described for use in promoting cognitive development in an infant born by C-section (Miles E A. Et al. Nutrients: 2017: 9(7):784; Hoff S. et al., Eur. J. Clin. Nutr.: 2005: 59: 1071-1080).

A link has been demonstrated between high intake of omega-3 PUFAs and increased incidence of allergic diseases. Because PUFAs are involved in cell signalling, gene expression and cellular function through membrane-mediated actions, PUFAs can modulate immune cell function, including inflammatory component. Ecosanoids, a proinflammatory molecules synthesised from 20 carbons PUFAs, are thought to be involved in clinical manifestations of allergic diseases (Miles E. A. et al., Nutrients:2017: 9(7):78). These proinflammatory molecules are counter interacted by the inflammation resolving molecules synthesized in the pathways of omega-3 fatty acids, therefore generating a biological plausible mechanism by which omega-3 PUFAs may protect against allergic sensitisation and manifestations. Some evidence suggests that a higher intake of n-3 PUFA is associated with a decreased risk of allergic sensitisation and allergic rhinitis (S. Hoff et al., Eur. J. Clinical Nutr.: 2005: 59: 1071-1080). Epidemiological evidence suggests that consumption of fish oil during pregnancy reduces allergic outcomes in infants and children.

Essential vitamins and minerals have long been known to play a role in innate and adaptive immune responses and therefore influence the development of allergic diseases in humans. Vitamins A and D has been extensively studied in preclinical models for their role in development of allergic diseases and show a promising result (Hufnagl K. et al., Allergo. J. Int.: 2018: 27(3):72-8; Matheu V. et al., Nutr. Metab.: 2009: 6: 44; Marmsjo K. et al., The American Journal of Clinical Nutrition: 2009: 90 (6): 1693-1698). Furthermore, children for whom an intake of multivitamin at or before the age of 4 years old was reported showed a decreased risk of sensitization to food allergens and tendencies toward inverse associations with allergic rhinitis (Marmsjo et al., the American J. Clinicl Nutr.: 2009: 90 (6): 1693-8).

Therefore, the intake by a C-section born child of a paediatric supplement or human milk fortifier composition that provides extra ALA, EPA and DHA (omega-3 fatty acids), essential vitamins and minerals may address the nutrient inadequacy in said child. Consequently, said child born via C-section may intake optimized amount of nutrients, thereby promoting immunity development, and/or reducing allergic outcome in said child.

In a still further aspect, the present invention provides for a paediatric supplement or human milk fortifier composition as herein described for use in promoting immunity development in child born via C-section.

In another aspect, the present invention provides for a paediatric supplement or human milk fortifier composition as described therein for use in reducing allergic outcome in a child born via C-section.

The paediatric supplement would be tailored or adapted to children born via C-section who are breast fed and the human milk fortifier according to the present invention may be tailored or adapted to fortify the breast milk of a women who has given birth via C-section. The purpose being to ensure that a child born via C-section does not receive less of one or more of the above-mentioned nutrients than a child born via vaginal delivery.

The human milk fortifier may be specifically tailored or adapted to supplement HM produced for child of at least 6 months of age, such as a child of 6 month of age, a child of 7 months of age, a child of 8 months of age, a child of 9 months of age, a child of 10 months of age, a child of 11 months of age, a child of 12 months of age. In another embodiment, the paediatric supplement or human milk fortifier is tailored of adapted for a child between 6 and 24 months of age, preferably for a child between 6 and 18 months of age, more preferably for a child between 6 and 12 months of age. The child may have been born via C-section.

The human milk fortifier may further comprise one or more ingredient selected from the group consisting of additional vitamins, additional minerals, protein, additional carbohydrates, and probiotics.

Further provided is a method of preparing a human milk fortifier composition tailored or adapted to fortify the breast milk of a women who has given birth via C-section, said method comprising the steps of: measuring out an appropriate amount of a human milk fortifier composition and mixing it with a diluent and/or additive. Then method may also comprise the step of determining whether the woman has given birth via C-section.

Also provided is a human milk fortifier as defined herein, for use in fortifying human breast milk and in particular human breast milk from a woman who has given birth via C-section.

The human milk fortifier as defined herein may provide an optimised amount of one or more nutrients selected in the group consisting of: ALA, DHA, EPA, 2′FL, LNnT, vitamin A, thiamine, thiamine monophosphate, vitamin B2, vitamin B6, vitamin B9, calcium and phosphorus, GD3, GM3 or any combination thereof, to a child.

The child may be selected from the group consisting of: a preterm child and a term child. In one embodiment, the child is a child born via C-section.

Also provided is a nutritional system comprising:
a. a human milk fortifier composition tailored to fortify the breast milk of women who have given birth via C-section, and
b. a human milk fortifier composition, e.g. a human milk fortifier tailored to fortify the breast milk of women who have given birth via vaginal delivery,
   wherein, said human milk fortifier composition tailored to fortify the breast milk of women who have given birth by caesarean section comprises one or more nutrient selected in the group consisting of: ALA, DHA, EPA, 2′FL, LNnT, vitamin A, thiamine, thiamine monophosphate, vitamin B2, vitamin B6, vitamin B9, calcium and phosphorus, GD3, GM3 or any combination thereof, in a concentration higher than in the human milk fortifier composition.

Also provided is a method of mitigating nutrient inadequacy in an infant born via C-section, said method comprising:
i) identifying the gap in certain nutrients between the HM composition from mothers who have delivered via C-section and the HM composition from mothers who have delivered vaginally;
ii) providing a paediatric supplement or human milk fortifier according to the invention to mitigate the identified nutritional inadequacies in a child born via C-section.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

### Definitions

The expressions “paediatric supplement” or “paediatric supplement composition” can be used interchangeably and refer to a composition which is intended to supplement the general diet of an infant. The paediatric supplement according to the present invention may be provided in different formats (such as drops, powder, oral solution) to be added to human breast milk or to be consumed as such.

The expressions “human milk fortifier” or “human milk fortifier composition” can be used interchangeably. They should be understood as comprising a nutritional composition for use in combination and in admixture with human breast milk. Unless otherwise specified, the expression “human milk fortifier composition” specifically excludes conventional infant formulas that provide the sole or primary source of infant nutrition and that are not typically combined and admixed with human milk to supplement human milk feedings.

Within the context of the present invention, the terms “nutrient inadequacy” indicates that the total intake of one or more nutrient in a child born via C-section is below (i.e. decreased) the total intake of those nutrient in child born via vaginal delivery because the amount of those one or more nutrients in HM produced by a woman who has delivered via C-section are decreased when compared to the amount of the same nutrients found in HM produced by mothers to children born via vaginal delivery.

Within the context of the present invention, the expression “prevent nutrient inadequacy” should be understood to include prevention of inadequacies of the nutrients in a child born via C-section, as well as reduction of the risk of nutrient inadequacies in HM of a woman who has delivered via C-section.

The term “fortifier” as used herein refers to a composition which comprises one or more nutrients having a nutritional benefit for children. A fortifier may be liquid or solid nutritional composition suitable for mixing with breast milk or formulas, for example with follow-on formula.

The term “supplement” refers to a foodstuff or a composition containing one or more nutrients intended to supplement the diet. A supplement may be liquid or solid (e.g. powder) composition and may be suitable for with breast milk or formulas, for example with follow-on formula.

The expressions “follow-up formula” or “follow-on formula” refers to a nutritional composition provided to a child from 6 months onwards.

The terms “fortify”, “fortifying”, “supplement”, “supplementing”, “complement” and “complementing” can be used interchangeably within the context of the present invention and should be understood as completing or supplementing the decreased amounts of one or more nutrients that were observed to be decreased in HM produced by mothers who have delivered via C-section when compared to the amounts of the same nutrients found in HM produced by mothers to infants born via vaginal delivery.

The terms “serving” or “dosage unit” within the context of the present invention indicates the amount of paediatric supplement or human milk fortifier which provided as an individual dose unit or which is to be consumed at an individual eating or feeding occasion according to the indication provided on the package of the paediatric supplement or human milk fortifier.

In an embodiment of the invention, the human milk fortifier composition is specifically tailored or adapted to fortify the breast milk of women who have given birth by C-section.

In another embodiment, the paediatric supplement is specifically tailored or adapted to a child born via C-section who is breast fed.

The term “C-section” as used herein refers to a caesarean section or caesarean delivery in general. C-section is the surgical procedure by which a baby is delivered through an incision in the mother's abdomen. The C-section may have been a planned/elective C-section, or an emergency C-section.

The term “infant” as used herein, refers to a human of less than about 6 months of age. The term includes preterm infants, premature infants, small for gestational age (SGA) infants and/or infant with low birth weight (LBW).

The expressions “preterm child” or “premature child” can be used interchangeably. They should be understood as comprising a child who is not born at term. Generally, they refer to infant born alive prior to 37 weeks of gestation/pregnancy.

The expression “small for gestational age infant” as used herein, refers to an infant who is smaller in size than normal for their gestational age at birth, most commonly defined as a weight below the 10th percentile for the gestational age. In some embodiments, SGA may be associated with intrauterine growth restriction (IUGR), which refers to a condition in which a foetus is unable to achieve its potential size.

The expression “low birth weight infant” as used herein refers to an infant that has a body weight under 2500 g at birth. It therefore encompasses:
an infant who has/had a body weight from 1800 to 2500 g at birth (usually called “low birth weight” or LBW)
an infant who has/had a body weight from 1000 to 1800 g at birth (called “very low birth weight” or VLBW)
an infant who has/had a body weight under 1000 g at birth (called “extremely low birth weight” or ELBW)

An infant or young child with low birth weight may or may not be preterm, and similarly, an infant or young child who is small for gestational age may or may not be preterm.

The term “child” as used herein, refers to a human from about 6 months to about 7 years of age, for example, between 6 months and 3 years of age. Preferably, the term child refers to a human age of at least 6 months.

The expression “sialylated oligosaccharide” as used herein refers to an oligosaccharide having a sialic acid (such as N-acetylneuraminic acid and/or N-glycolylneuraminic acid) residue.

The expression “N-acetylatedoligosaccharide” as used herein refers to an oligosaccharide having at least one hexose carrying an N-acetyl residue.

The expression “fucosylated oligosaccharide” as used herein refers to an oligosaccharide having a fucose residue.

The terms “promote”, “promotion” and “promoting” can be used interchangeably. They should be understood as comprising support or help to the health in an individual, for example to support or help the development or growth of an individual. The individual may not suffer from a disease but may be susceptible to the development of unhealthy conditions, for example later in life.

The terms “treat”, “treating” or “treatment” as used therein may mean both prophylactic or preventive treatment (i.e. that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder. It may include treatment of patients at risk of contracting a disease or suspected to have contacted a disease, as well as patients who are ill or have been diagnosed as suffering from a disease or a medical condition. The terms “treat”, “treating” or “treatment” do not necessarily imply that a subject is treated until total recovery. The terms “treat”, “treating” or “treatment” also refer to the maintenance and/or promotion of health in an individual not suffering from a disease but who may be susceptible to the development of unhealthy condition. The terms “treat”, “treating” or “treatment” are also intended to include potentiation or otherwise enhancement of one or more prophylactic or therapeutic measures. As an embodiment, the term “treatment” indicates prophylactic or preventive treatment.

Within the context of the present invention, the expression “optimized gut microbiota” means a healthy gut microbiota that has developed stable microbiome diversity, i.e. it contains many different types of microbial species (bacterial or otherwise), said microbial species having the ability to resist to change in the setting of an ecological stress (resistance) or to return to an equilibrium state following a stress-related perturbation (resilience). The microbiota (i.e. the microbial community) consists in the collection of microorganisms, composed of archaea, bacteria and eukarya, colonizing the human gastrointestinal tract. Disruption of the microbiota may therefore impact the microbiome and eventually result in impaired growth or pathogenesis in the individual.

Within the context of the present invention, the expressions “in the prevention and/or treatment of inflammatory process”, “to prevent and/or treat inflammatory process” or “for the prevention and/or the treatment of inflammatory process” can be used interchangeably. They should be understood as comprising the decrease of the duration of inflammatory process, of the severity of inflammatory process. These expressions also encompass the relieve of the symptoms induced by inflammatory process, such as pain, stress, tiredness, and/or the decrease of complications caused by inflammatory process. The term “inflammatory process” can be understood as an inflammatory response (i.e. inflammation) that may occur when tissues are injured by bacteria, trauma, toxins, heat or any other cause.

Within the context of the present invention, the expression “cognitive development” should be understood as comprising how an individual, for example an infant or a child, learns to think, reason, and use language. It is the development of knowledge, skills, problem solving and dispositions, which help individuals to think about and understand the world around them. Among the areas of cognitive development are information processing, intelligence, reasoning, language development and memory. Infants are aware of their surroundings and show interest in the exploration of their environment from the time they are born. From birth, the infants start to actively learn, to gather, sort and process information from their surroundings using the data to develop perception and thinking skills.

Within the context of the present invention, the expression “immunity development” as used herein refers to the development of the immune system by adaptation in response to antigen stimulus, which starts at birth and continues throughout life. Immunity is developing and still to reach maturity in infants and children.

Within the context of the present invention, the term “allergy” comprises food allergy, atopic dermatitis, allergic asthma, and/or allergic rhinitis. For example, allergy is a food allergy.

### The Paediatric Supplement or Human Breast Milk Fortifier Composition

In a first aspect of the present invention there is provided a paediatric supplement or a human milk fortifier composition comprising at least one nutrient selected in the group consisting of: ALA, DHA, EPA, 2′FL, LNnT, vitamin A, thiamine, thiamine monophosphate, vitamin B2, vitamin B6, vitamin B9, calcium and phosphorus, GD3, GM3 or any combination thereof.

In one embodiment, the paediatric supplement or the human milk fortifier composition is tailored or adapted to supplement or fortify the breast milk of a woman who has given birth via C-section. A paediatric supplement or human milk fortifier composition, as disclosed herein, may be considered as specifically tailored or adapted to a child born via C-section who is breast fed or specifically tailored or adapted to fortify the breast milk of a woman who has given birth via C-section if it comprises at least one nutrient selected in the group consisting of: ALA, DHA, EPA, 2′FL, LNnT, vitamin A, thiamine, thiamine monophosphate, vitamin B2, vitamin B6, vitamin B9, calcium and phosphorus, GD3, GM3, as described herein. Said paediatric supplement or human milk fortifier composition may, for example, comprise said at least one or more nutrient selected in the group consisting of: ALA, DHA, EPA, 2′FL, LNnT, vitamin A, thiamine, thiamine monophosphate, vitamin B2, vitamin B6, vitamin B9, calcium and phosphorus, GD3, GM3, in an amount sufficient to address the deficiency of such at least one nutrient in the human breast milk of mothers who have given birth via C-section in comparison to mothers who have given birth vaginally. A sufficient amount of a nutrient may for example be an amount equal to or greater than an amount that a child born via vaginal delivery would receive, or may for example, be any amount that is equal to or higher than the difference found in the concentration, e.g. averages, in human milk produced by women who have given birth by vaginal delivery and women who have given birth via C-section delivery. Said paediatric supplement or human milk fortifier composition may be a delivery mode specific paediatric supplement or human milk fortifier. For example, said paediatric supplement may be sold for use in children born via C-section. Said paediatric supplement may be marketed as being for use to to supplement the general diet of a child born via C-section. For example, said human milk fortifier may be sold specifically for use in women who have given birth via C-section. Said human milk fortifier may be marketed as a being for use to fortify the breast milk of women who have given birth via C-section.

In one embodiment of the present invention, the paediatric supplement or human breast milk fortifier composition of the present invention comprises at least one nutrient selected in the group consisting of: ALA, DHA, EPA, 2′FL, LNnT, vitamin A, thiamine, thiamine monophosphate, vitamin B2, vitamin B6, vitamin B9, calcium and phosphorus, GD3, GM3, respectively in the amounts reported in Table I below.

TABLE I

| Nutrients | Daily amount to be supplemented | |
|---|---|---|
| ALA (18:3 n-3) | 19-80 | mg |
| DHA (22:6 n-3) | 13-25 | mg |
| EPA (20:5n-3) | 6-9 | mg |
| 2′FL | 280-1050 | mg |
| LNnT | 30-50 | mg |
| Vitamin A | 224-342 | μg |
| Thiamine | 6.6-10 | μg |
| (vitamin b1) | | |
| Thiamine | 10-15 | μg |
| monophosphate | | |
| Vitamin B2 | 9-19 | μg |
| (Riboflavin) | | |
| Vitamin B6 | 15-68 | μg |
| Vitamin B9 | 1.1-9 | μg |
| Calcium | 10-16 | mg |
| Phosphorus | 4.5-14 | mg |
| GD₃ | 8-12 | mg |
| GM₃ | 1-1.5 | mg |

In one embodiment, the paediatric supplement or human breast milk fortifier composition according to the present invention may be administered in 1, 2, 3 or 4 daily servings or dosage units to provide the total daily amounts of the nutrients as above described. As it will be apparent to a person skilled in the art, to calculate the amount of each nutrient contained in each serving or dosage unit of the paediatric supplement or human breast milk fortifier composition according to the present invention, the daily amount as above reported will be divided by 1, 2, 3 or 4 respectively.

The concentrations listed herein, when expressed as mg/mL, refer to concentrations after a composition has been reconstituted or mixed with water or milk.

In an embodiment of the present invention the paediatric supplement or human milk fortifier composition is tailored or adapted for child of at least 6 months of age, such as a child of 6 month of age, a child of 7 months of age, a child of 8 months of age, a child of 9 months of age, a child of 10 months of age, a child of 11 months of age, a child of 12 months of age. In another embodiment, the paediatric supplement or human milk fortifier is tailored of adapted for a child between 6 and 24 months of age, preferably for a child between 6 and 18 months of age, more preferably for a child between 6 and 12 months of age.

In one embodiment, the child is selected from the group consisting of preterm children and term children.

In an embodiment of the present invention, the paediatric supplement or human milk fortifier composition is tailored or adapted for a child of at least 6 months of age and said composition comprises one or more nutrient selected in the group consisting of: ALA, DHA, EPA, 2′FL, LNnT, vitamin A, thiamine, thiamine monophosphate, vitamin B2, vitamin B6, vitamin B9, calcium and phosphorus, GD3, GM3, or any combination thereof.

In one embodiment of the present invention, it is provided a paediatric supplement or human milk fortifier composition comprising at least one nutrient selected in the group consisting of:
alpha linolenic acid (ALA) in a daily amount ranging from 19 to 80 mg;
docosahexaenoic acid (DHA) in a daily amount ranging from 13 to 25 mg;
eicosapentanoic acid (EPA) in a daily amount ranging from 6 to 9 mg;
2-fucosyl lactose (2′-FL) in a daily amount ranging from 280 to 1050 mg;
LNnT in a daily amount ranging from 30 to 50 mg;
vitamin A in a daily amount ranging from 224 to 342 μg;
thiamine in a daily amount ranging from 6.6 to 10 μg;
thiamine monophosphate in a daily amount ranging from 10 to 15 μg;
vitamin B2 in a daily amount ranging from 9 to 19 μg;
vitamin B6 in a daily amount ranging from 15 to 68 μg;
vitamin B9 in a daily amount ranging from 1.1 to 9 μg;
calcium in a daily amount ranging from 10 to 16 mg;
phosphorus in a daily amount ranging from 4.5 to 14 mg;
GD3 in a daily amount ranging from 8 to 12 mg; and
GM3 in a daily amount ranging from 1 to 1.5 mg;
   or any combination thereof.

In a further embodiment, the present invention provides a paediatric supplement or human milk fortifier composition as above described wherein the composition comprises:
alpha linolenic acid (ALA) in a daily amount ranging from 19 to 80 mg;
docosahexaenoic acid (DHA) in a daily amount ranging from 13 to 25 mg;
eicosapentanoic acid (EPA) in a daily amount ranging from 6 to 9 mg;
2-fucosyl lactose (2′-FL) in a daily amount ranging from 280 to 1050 mg;
   and which optionally comprises at least one nutrient selected in the group consisting of:
LNnT, for example in a daily amount ranging from 30 to 50 mg;
vitamin A, for example in a daily amount ranging from 224 to 342 μg;
thiamine, for example in a daily amount ranging from 6.6 to 10 μg;
thiamine monophosphate, for example in a daily amount ranging from 10 to 15 μg;
vitamin B2, for example in a daily amount ranging from 9 to 19 μg;
vitamin B6, for example in a daily amount ranging from 15 to 68 μg;
vitamin B9, for example in a daily amount ranging from 1.1 to 9 μg;
calcium, for example in a daily amount ranging from 10 to 16 μg;
phosphorus, for example in a daily amount ranging from 4.5 to 14 mg;
GD3, for example in a daily amount ranging from 8 to 12 mg; and
GM3, for example in a daily amount ranging from 1 to 1.5 mg;
   or any combinations thereof.

In another embodiment, the present invention provides fora paediatric supplement or human milk fortifier composition as herein described which comprises at least one nutrient selected in the group consisting of
calcium, for example in a daily amount ranging from 10 to 16 mg;
phosphorus, for example in a daily amount ranging from 4.5 to 14 mg;
GD3, for example in a daily amount ranging from 8 to 12 mg; and
GM3, for example in a daily amount ranging from 1 to 1.5 mg;
   and
LNnT, for example in a daily amount ranging from 30 to 50 mg;
   and optionally comprises at least one nutrient selected in the group consisting of:
alpha linolenic acid (ALA), for example in a daily amount ranging from 19 to 80 mg;
docosahexaenoic acid (DHA), for example in a daily amount ranging from 13 to 25 mg;
eicosapentanoic acid (EPA), for example in a daily amount ranging from 6 to 9 mg;
2-fucosyl lactose (2′-FL), for example in a daily amount ranging from 280 to 1050 mg;
vitamin A, for example in a daily amount ranging from 224 to 342 μg;
thiamine, for example in a daily amount ranging from 6.6 to 10 μg;
thiamine monophosphate, for example in a daily amount ranging from 10 to 15 μg;
vitamin B2, for example in a daily amount ranging from 9 to 19 μg;
vitamin B6, for example in a daily amount ranging from 15 to 68 μg;
vitamin B9, for example in a daily amount ranging from 1.1 to 9 μg;
   or any combination thereof.

The paediatric supplement or human milk fortifier composition of the invention can also comprise any other ingredients or excipients known to be employed in human milk fortifier compositions or paediatric supplements.

Non limiting examples of such ingredients include proteins, amino acids, carbohydrates, lipids, prebiotics or probiotics, essential fatty acids, nucleotides, nucleosides, vitamins, minerals and other micronutrients.

In an embodiment of the invention the paediatric supplement or human milk fortifier composition further comprises one or more ingredients selected from the group consisting of additional vitamins, additional minerals, protein, additional carbohydrates, and probiotics.

Non limiting examples of proteins include casein, alpha-lactalbumin, whey, soy protein, rice protein, corn protein, oat protein, barley protein, wheat protein, rye protein, pea protein, egg protein, sunflower seed protein, potato protein, fish protein, meat protein, lactoferrin, serum albumin, immunoglobins, and combinations thereof.

Non limiting examples of amino acids include leucine, threonine, tyrosine, Isoleucine, arginine, alanine, histidine, isoleucine, proline, valine, cysteine, glutamine, glutamic acid, glycine, serine, arginine, lysine, methionine, phenylalanine, tryptophane, asparagine, aspartic acid, and combinations thereof.

Non limiting examples of digestible carbohydrates include lactose, saccharose, maltodextrin, starch, and combinations thereof.

Non limiting examples of lipids include palm olein, high oleic sunflower oil, high oleic safflower oil, canola oil, fish oil, coconut oil, bovine milk fat, and combinations thereof.

Non limiting examples of essential fatty acids include linoleic acid (LA) and polyunsaturated fatty acids (PUFAs). The compositions of the invention may further contain phospholipids such as sphingomyelin, phospholipids phosphatidylcholine, phosphatidylethanolamine, phosphatidylinositol, phosphatidylserine, and combinations thereof.

None limiting examples of non-digestible carbohydrates (prebiotics) include oligosaccharides optionally containing additional HMOs, fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

The additional HMO may be a sialylated oligosaccharide, a fucosylated oligosaccharide, an N-acetylated oligosaccharide, or any combination thereof. The one or more HMO may for example be selected from the group consisting of; 3′-fucosyllactose, 3′-sialyllactose, 6′-galactosyllactose, difucosyllacto-N-Hexose-a, fucosyllacto-N-hexose-III, Lacto-N-fucosylpentaose-I, Lacto-N-fucosylpentaose-III, Lacto-N-fucosylpentaose-V, Lacto-N-hexaose (A), Lacto-N-Neodifucosylhexaose, Lacto-N-Neofucosylpentaose, Lacto-N-Tetraose, and any combination thereof.

The human milk fortifier composition may comprise an additional HMO in a range of 0.1 to 10000 mg/L.

Non limiting examples of probiotics include Bifidobacterium, Lactobacillus, Lactococcus, Enterococcus, Streptococcus, Kluyveromyces, Saccharoymces, Candida, in particular selected from the group consisting of Bifidobacterium longum ssp longum, Bifidobacterium lactis, Bifidobacterium animalis, Bifidobacterium breve, Bifidobacterium longum ssp infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactobacillus reuteri protectis, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM I-2618), Bifidobacterium longum NCC490 (CNCM I-2170), Bifidobacterium lactis NCC2818 (CNCM I-3446), Bifidobacterium breve strain A, Lactobacillus paracasei NCC2461 (CNCM I-2116), Lactobacillus johnsonii NCC533 (CNCM I-1225), Lactobacillus rhamnosus GG (ATCC53103), Lactobacillus rhamnosus NCC4007 (CGMCC 1.3724), Enterococcus faecium SF 68 (NCC2768; NCIMB10415), and combinations thereof.

In some embodiments, the paediatric supplement or human milk fortifier composition according to the present invention comprises one or more probiotics selected from the group consisting of Bifidobacterium longum ssp longum, Bifidobacterium lactis, Bifidobacterium animalis, Bifidobacterium breve, Bifidobacterium longum ssp infantis, Bifidobacterium adolescentis.

In some embodiments, at least one, or at least two or more probiotics are included in the paediatric supplement or human milk fortifier composition according to the present invention in amount from about 10¹ to 10¹⁸ cfu (colony forming unit), such as 10² to 10¹⁵, 10³ to 10¹² 10⁵ to 10¹², 10⁶ to 10¹², 10⁷ to 10¹², 10⁸ to 10¹² or 10⁹ to 10¹² cfu of each strain per g on a dry weight basis or per mL of the composition on a volume basis. In some embodiments, the human milk fortifier composition comprises 10¹, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, or about 10¹⁸ cfu of one or more probiotics per g on a dry weight basis or per mL of the composition on a volume basis. In some other embodiments, the human milk fortifier composition comprises about 10¹ to about 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, or about 10¹⁸ cfu of one or more probiotics per g on a dry weight basis or per mL of the composition on a volume basis.

Non limiting examples of nucleotides include cytidine monophosphate (CMP), uridine monophosphate (UMP), adenosine monophosphate (AMP), guanosine monophosphate (GMP), and combinations thereof.

Non limiting examples of additional vitamins and minerals include vitamin Bi2, vitamin E, vitamin K, vitamin C, vitamin D, folic acid, inositol, niacin, biotin, pantothenic acid, choline, iodine, iron, magnesium, copper, zinc, manganese, chloride, potassium, sodium, selenium, chromium, molybdenum, taurine, L-carnitine, and combinations thereof. Minerals are usually added in salt form.

Other suitable and desirable ingredients of paediatric supplement or human milk fortifier composition, that may be employed in the compositions of the invention, are described in guidelines issued by the Codex Alimentarius.

The paediatric supplement of the invention may be prepared in any way known in the art to prepare paediatric supplements.

It is well within the purview of the skilled person to decide on a method depending on the type of paediatric supplement in question e.g. powder or liquid.

The human milk fortifier composition of the invention may be prepared in any way known in the art to prepare human milk fortifier compositions.

It is well within the purview of the skilled person to decide on a method depending on the type of human milk fortifier in question e.g. powder or liquid. An exemplary method for preparing a human milk fortifier in accordance with the invention is set out below.

A human milk fortifier may be prepared, for example, by blending together lipid, protein, HMO, and other carbohydrates in appropriate proportions. If used, emulsifiers may be included in the blend at this stage. The vitamins and minerals may be added at this stage but are usually added later to avoid thermal degradation. Any lipophilic vitamins, such as vitamin A, D, E and K, and emulsifiers may be dissolved into the fat source prior to blending. Water, preferably water which has been subjected to reverse osmosis, may then be mixed to provide a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger, for example a plate heat exchanger.

The liquid mixture may then be cooled to about 60° C. to about 85° C., for example by flash cooling. The liquid mixture may then be homogenised, for example in two stages at about 7 MPa to about 40 MPa in the first stage and about 2 MPa to about 14 MPa in the second stage. The homogenised mixture may then be further cooled and any heat sensitive components, such as vitamins and minerals may be added. The pH of the homogenised mixture is conveniently standardised at this point.

The homogenized liquid mixture is then filled into suitable containers, preferably aseptically. However, the liquid composition may also be reported in the container. Suitable apparatus for carrying out filling of this nature is commercially available.

A human milk fortifier composition specifically tailored or adapted to fortify the breast milk of a women who has given birth via caesarean section may be prepared from a human milk fortifier composition e.g. a human milk fortifier composition not specifically tailored to fortify the breast milk of a woman who has given birth via a particularly delivery mode e.g. C-section or vaginal delivery.

Accordingly, in another aspect of the present invention there is provided a method of preparing a human milk fortifier composition tailored or adapted to fortify the breast milk of a women who has given birth via C-section, said method comprising the steps of: measuring out an appropriate amount of a human milk fortifier composition e.g. a human milk fortifier composition not specifically tailored to fortify the breast milk of a woman who has given birth by a particularly delivery mode, and mixing it with an additive and/or a diluent e.g. one or more nutrients selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3 and/or water, so as to arrive at a human milk fortifier composition tailored or adapted to fortify the breast milk of a women who has given birth via C-section in accordance with the invention.

The additive may be a one or more nutrient selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3 in a concentration such, that when the additive is mixed with a human milk fortifier composition, and optionally a diluent, the resulting mixture is a human milk fortifier tailored or adapted to fortify the breast milk of a women who has given birth via C-section, in accordance with the invention.

The additive may be a delivery mode specific additive e.g. an additive marketed as specifically being for use by women who have given birth via C-section.

In another aspect of the present invention there is provided a paediatric supplement or human milk fortifier composition in accordance with the invention, for use in supplementing or fortifying human breast milk.

In an embodiment the human breast milk is breast milk from women who have given birth via C-section.

In one aspect of the present invention, there is provided a paediatric supplement or human milk fortifier composition in accordance with the invention, for use in providing an adequate nutrition to a child born via C-section. The paediatric supplement or human milk fortifier composition in accordance with the invention is tailored or adapted to complete or supplement the decreased amounts of one or more nutrients that were observed to be decreased in HM produced by mothers who have delivered by C-section, said one or more nutrients being selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3.

In another aspect of the present invention there is provided the use of a paediatric supplement or a human milk fortifier composition to provide an adequate nutrition to a child born via C-section.

In another aspect of the present invention there is provided a paediatric supplement or human milk fortifier composition in accordance with the invention, for use to provide an optimised amount and/or to prevent the sub-optimal intake of one or more nutrients selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3 to an infant born by C-section. An optimised amount of one or more nutrients selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3 would be an amount equal to or greater than an amount e.g. the average amount, that an child born by vaginal delivery would be considered to receive e.g. an amount of DHA set out in Table I included herein.

In another aspect of the present invention there is provided a paediatric supplement or human milk fortifier composition in accordance with the invention, for use in preventing sub-optimal growth and development in a child born via C-section. In one embodiment, there is provided a paediatric supplement or human milk fortifier composition in accordance with the invention, for use in optimising the health and development and/or preventing the sub-optimal health and development e.g. growth and development of a child born via C-section.

The human milk fortifier compositions of the invention may not only optimise the health and development of a child born via C-section short term but may also do so in the long term.

In another aspect of the present invention there is provided a paediatric supplement or human milk fortifier composition in accordance with the invention, for use in the prevention and/or treatment of inflammatory process in a child born via C-section.

In another aspect of the present invention there is provided a paediatric supplement or human milk fortifier composition in accordance with the invention, for use in optimising the gut microbiota and/or preventing sub-optimal gut microbiota in a child born via C-section.

In another aspect of the present invention there is provided the use of paediatric supplement or of a human milk fortifier composition in accordance with the invention, in the manufacture of a composition for use in optimising the gut microbiota in a child born via C-section.

A non-optimal gut microbiota may be one showing presence of one or several pathobionts and/or opportunistic pathobionts and/or their toxins and/or virulence factors and/or antibiotic resistance genes. An optimal gut microbiota may be one not showing presence of one or several pathobionts and/or opportunistic pathobionts and/or their toxins and/or virulence factors and/or antibiotic resistance genes.

The paediatric supplement human milk fortifier compositions of the invention may not only optimise the gut microbiota composition short term but may also do so in the long term.

Long term effects may only be evident in months or years e.g. 6 months, 9 months, 12 months, 5 years, 10 years, or 20 years

In another aspect of the present invention there is provided a paediatric supplement or human milk fortifier composition in accordance with the invention, for use in promoting cognitive development in a child born via C-section.

In another aspect of the present invention there is provided a paediatric supplement or human milk fortifier composition in accordance with the invention, for use in promoting immunity development in a child born via C-section.

In one aspect of the present invention there is provided the use of a paediatric supplement or a human milk fortifier composition in accordance with the invention to supplement or fortify human breast milk. Preferably, the human breast milk is from a woman who has given birth via C-section.

In another aspect of the present invention there is provided the use of a paediatric supplement or a human milk fortifier composition in accordance with the invention, to supplement or fortify human breast milk and to improve/prevent sub-optimal breast milk quality wherein said breast milk is from a woman who has given birth via C-section. The quality of breast milk in a woman who has given birth by C-section may be considered sub-optimal if it comprises one or more nutrients selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3 in a concentration less than the concentration found in breast milk from a woman who has given birth vaginally e.g. in a concentration less than the average found in woman who have given birth vaginally.

In another aspect of the present invention there is provided the use of a paediatric supplement or human milk fortifier according to the invention to optimise and/or prevent the sub-optimal health and development and/or gut flora composition in a child born via C-section.

In another aspect of the present invention there is provided the use of a paediatric supplement or a human milk fortifier composition to prevent and/or treat inflammatory process, to optimize the gut microbiota and/or prevent sub-optimal gut microbiota, to promote cognitive development and/or to promote immunity development in a child born via C-section.

Health and development and/or gut flora composition may be optimised short term or long term.

A human milk fortifier tailored or adapted to fortify the breast milk of a woman who has given birth via C-section may be included in a nutritional system.

The term “nutritional system” as used herein refers to a collection of more than one synthetic nutritional compositions advertised or sold as part of the same product range e.g. a collection of human milk fortifiers and/or follow-on formulas sold under the same brand and tailored or adapted to the nutritional needs of child born via different delivery modes e.g. C-section or vaginally. The synthetic nutritional compositions making up the nutritional system may be packaged individually e.g. in capsules or boxes. Said packages can be sold individually, grouped together e.g. wrapped by plastic film or combined in a box, or in a combination of these two ways. The nutritional system may also comprise synthetic nutritional compositions for children older than 12 months.

In a further aspect of the present invention there is provided a nutritional system comprising:
a. a human milk fortifier composition tailored to fortify the breast milk of women who have given birth via C-section, in accordance with the invention, and
b. a human milk fortifier composition e.g. a human milk fortifier composition not specifically tailored to fortify the breast milk of a woman who has given birth via a particular delivery mode,
   wherein said human milk fortifier composition tailored to fortify the breast milk of women who have given birth via C-section comprises one or more nutrients selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3 in a concentration higher than in the human milk fortifier composition e.g. human milk fortifier composition not specifically tailored to fortify the breast milk of a woman who has given birth via a particularly delivery.

The concentration of one or more nutrients selected in the list consisting of: ALA, DHA, EPA, 2′FL, LNnT, vitamin A, thiamine, thiamine monophosphate, vitamin B2, vitamin B6, vitamin B9, calcium and phosphorus, GD3, GM3 in the human milk fortifier tailored or adapted for a woman who has given birth via C-section may be higher by any amount.

### Nutrients

### Alpha Linolenic Acid (ALA)

In one embodiment of the present invention, a paediatric supplement or human milk fortifier composition is provided which comprises ALA.

ALA may be incorporated in the composition of the invention as such or in the form of a physiologically acceptable derivative, such as a salt and/or by any source comprising ALA or any mixture thereof, such as oils enriched in ALA, seeds and nuts. In one embodiment, ALA is provided to the composition in the form of an oil enriched in such fatty acid.

As it is evident to the person skilled in the art, different ingredients may provide different amounts of ALA in the composition according to the present invention, depending on the nature and amount of the ingredient used. It will be nonetheless routine work to the skilled person to calculate the amount of ingredient needed to provide the claimed amount of ALA, based on the specification of the specific ingredient provided by the supplier.

In one embodiment, the paediatric supplement or human milk fortifier composition of the present invention comprises ALA in an amount of at least 19 mg/day. In another embodiment, the composition of the present invention comprises ALA in an amount ranging from 19 to 80 mg/day.

In one embodiment, the paediatric supplement or human milk fortifier composition of the present invention comprises ALA in an amount of at least 19 mg. In another embodiment, the paediatric supplement or human milk fortifier composition of the present invention comprises ALA in an amount ranging from 19 to 80 mg.

In such embodiment, the paediatric supplement or human milk fortifier composition of the present invention delivers in one serving or dosage unit the daily amount of ALA considered necessary to fill the gap of ALA between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of ALA as above described. In such embodiment, as it will be apparent to a person skilled in the art, the amount of ALA contained in each serving of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Docosahexaenoic Acid (DHA)

In one embodiment of the present invention, a paediatric supplement or human milk fortifier composition is provided which comprises DHA.

DHA may be incorporated in the paediatric supplement or human milk fortifier composition of the invention as DHA or in the form of a physiologically acceptable derivative, such as a salt and/or by any source comprising DHA or any mixture thereof, such as for example oils enriched in DHA, algae or fish oil. In one embodiment, DHA is provided to the paediatric supplement or human milk fortifier composition in the form of an oil enriched in such fatty acid.

As it is evident to the person skilled in the art, different ingredients may provide different amounts of DHA in the paediatric supplement or human milk fortifier composition according to the present invention, depending on the nature and amount of the ingredient used. It will be nonetheless routine work to the skilled person to calculate the amount of ingredient needed to provide the claimed amount of DHA, based on the specification of the specific ingredient provided by the supplier.

In one embodiment, the paediatric supplement or human milk fortifier composition of the present invention comprises DHA in an amount of at least 13 mg/day. In another embodiment, the paediatric supplement or human milk fortifier composition of the present invention comprises DHA in an amount ranging from 13 to 25 mg/day.

In one embodiment, the paediatric supplement or human milk fortifier composition of the present invention comprises DHA in an amount of at least 13 mg. In another embodiment, the paediatric supplement or human milk fortifier composition of the present invention comprises DHA in an amount ranging from 13 to 25 mg.

In such embodiment, the paediatric supplement or human milk fortifier composition of the present invention delivers in one serving or dosage unit, the daily amount of DHA considered necessary to fill the gap of DHA between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of DHA as above described. In such embodiment, as it will be apparent to a person skilled in the art, the amount of DHA contained in each serving of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Eicosapentanoic Acid (EPA)

In one embodiment of the present invention, a supplement or human milk fortifier composition is provided which comprises EPA.

EPA may be incorporated in the paediatric supplement or human milk fortifier composition of the invention as EPA or in the form of a physiologically acceptable derivative, such as a salt and/or by any source comprising EPA or any mixture thereof, such as for example oils enriched in EPA, algae or fish oil. In one embodiment, EPA is provided to the paediatric supplement or human milk fortifier composition in the form of an oil enriched in such fatty acid.

As it is evident to the person skilled in the art, different ingredients may provide different amounts of EPA in the paediatric supplement or human milk fortifier composition according to the present invention, depending on the nature and amount of the ingredient used. It will be nonetheless routine work to the skilled person to calculate the amount of ingredient needed to provide the claimed amount of EPA, based on the specification of the specific ingredient provided by the supplier.

In one embodiment, the paediatric supplement or human milk fortifier composition of the present invention comprises EPA in an amount of at least 6 mg/day. In another embodiment, the paediatric supplement or human milk fortifier composition of the present invention comprises EPA in an amount ranging from 6 to 9 mg/day.

In one embodiment, the paediatric supplement or human milk fortifier composition of the present invention comprises EPA in an amount of at least 6 mg. In another embodiment, the paediatric supplement or human milk fortifier composition of the present invention comprises EPA in an amount ranging from 6 to 9 mg.

In such embodiment, the paediatric supplement or human milk fortifier composition of the present invention delivers in one serving the daily amount of EPA considered necessary to fill the gap of EPA between the human breast milk of a mother who delivered vaginally and that of a mother who delivered by C-section.

In one embodiment, the paediatric supplement or human milk fortifier composition according to the present invention may be administered in 1, 2, 3 or 4 daily servings to provide the total daily amounts of EPA as above described. In such embodiment, as it will be apparent to a person skilled in the art, the amount of EPA contained in each serving of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### GD3

In one embodiment of the present invention, a supplement or human milk fortifier composition is provided which comprises GD3.

GD3 may be incorporated in the paediatric supplement or human milk fortifier composition of the invention as such or in the form of a physiologically acceptable derivative, such as a salt and/or by any source comprising GD3 or any mixture thereof, such as dairy ingredients (such as milk fat globule membrane and whey protein), meat, fish, animal derived ingredients. In one embodiment, GD3 is provided as such to the paediatric supplement or human milk fortifier composition.

As it is evident to the person skilled in the art, different ingredients may provide different amounts of GD3 in the paediatric supplement or human milk fortifier composition according to the present invention, depending on the nature and amount of the ingredient used. It will be nonetheless routine work to the skilled person to calculate the amount of ingredient needed to provide the claimed amount of GD3, based on the specification of the specific ingredient provided by the supplier.

In one embodiment, the paediatric supplement or human milk fortifier composition of the present invention comprises GD3 in an amount of at least 8 mg/day. In another embodiment, the composition of the present invention comprises GD3 in an amount ranging from 8 to 12 mg/day.

In one embodiment, the paediatric supplement or human milk fortifier composition of the present invention comprises GD3 in an amount of at least 8 mg. In another embodiment, the paediatric supplement or human milk fortifier composition of the present invention comprises GD3 in an amount ranging from 8 to 12 mg.

In such embodiment, the paediatric supplement or human milk fortifier composition of the present invention delivers in one serving the daily amount of GD3 considered necessary to fill the gap of GD3 between the human breast milk of a mother who delivered vaginally and that of a mother who delivered by C-section.

In one embodiment, the paediatric supplement or human milk fortifier composition according to the present invention may be administered in 1, 2, 3 or 4 daily servings to provide the total daily amounts of GD3 as above described. In such embodiment, as it will be apparent to a person skilled in the art, the amount of GD3 contained in each serving of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### GM3

In one embodiment of the present invention, a supplement or human milk fortifier composition is provided which comprises GM3.

GM3 may be incorporated in the paediatric supplement or human milk fortifier composition of the invention as folic acid or in the form of a physiologically acceptable derivative, such as a salt and/or by any source comprising GM3 or any mixture thereof, such as dairy ingredients (such as milk fat globule membrane and whey protein), meat, fish, animal derived ingredients). In one embodiment, GM3 is provided as such to the paediatric supplement or human milk fortifier composition.

As it is evident to the person skilled in the art, different ingredients may provide different amounts of GM3 in the paediatric supplement or human milk fortifier composition according to the present invention, depending on the nature and amount of the ingredient used. It will be nonetheless routine work to the skilled person to calculate the amount of ingredient needed to provide the claimed amount of GM3, based on the specification of the specific ingredient provided by the supplier.

In one embodiment, the paediatric supplement or human milk fortifier composition of the present invention comprises GM3 in an amount of at least 1 mg/day. In another embodiment, the paediatric supplement or human milk fortifier composition of the present invention comprises GM3 in an amount ranging from 1 to 1.5 mg/day.

In one embodiment, the paediatric supplement or human milk fortifier composition of the present invention comprises GM3 in an amount of at least 1 mg. In another embodiment, the paediatric supplement or human milk fortifier composition of the present invention comprises GM3 in an amount ranging from 1 to 1.5 mg.

In such embodiment, the paediatric supplement or human milk fortifier composition of the present invention delivers in one serving the daily amount of GM3 considered necessary to fill the gap of GM3 between the human breast milk of a mother who delivered vaginally and that of a mother who delivered by C-section.

In one embodiment, the paediatric supplement or human milk fortifier composition according to the present invention may be administered in 1, 2, 3 or 4 daily servings to provide the total daily amounts of GM3 as above described. In such embodiment, as it will be apparent to a person skilled in the art, the amount of GM3 contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### 2-Fucosyllactose (2′-FL)

In one embodiment of the present invention, a supplement or human milk fortifier composition is provided which comprises 2′-FL.

2′-FL may be incorporated in the paediatric supplement or human milk fortifier composition of the invention as such or in the form of a physiologically acceptable derivative, such as a salt and/or by any source comprising 2′-FL or any mixture thereof.

As it is evident to the person skilled in the art, different ingredients may provide different amounts of 2′-FL in the paediatric supplement or human milk fortifier composition according to the present invention, depending on the nature and amount of the ingredient used. It will be nonetheless routine work to the skilled person to calculate the amount of ingredient needed to provide the claimed amount of 2′-FL, based on the specification of the specific ingredient provided by the supplier.

In one embodiment, the paediatric supplement or human milk fortifier composition of the present invention comprises 2′-FL in an amount of at least 280 mg/day. In another embodiment, the paediatric supplement or human milk fortifier composition of the present invention comprises 2′-FL in an amount ranging from 280 to 1050 mg/day.

In one embodiment, the paediatric supplement or human milk fortifier composition of the present invention comprises 2′-FL in an amount of at least 280 mg. In another embodiment, the paediatric supplement or human milk fortifier composition of the present invention comprises 2′-FL in an amount ranging from 280 to 1050 mg.

In such embodiment, the paediatric supplement or human milk fortifier composition of the present invention delivers in one serving the daily amount of 2′-FL considered necessary to fill the gap of 2′-FL between the human breast milk of a mother who delivered vaginally and that of a mother who delivered by C-section.

In one embodiment, the paediatric supplement or human milk fortifier composition according to the present invention may be administered in 1, 2, 3 or 4 daily servings to provide the total daily amounts of 2′-FL as above described. In such embodiment, as it will be apparent to a person skilled in the art, the amount of 2′-FL contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Lacto-N-Neotetraose (LNnT)

In one embodiment of the present invention, a supplement or human milk fortifier composition is provided which comprises LNnT.

LNnT may be incorporated in the paediatric supplement or human milk fortifier composition of the invention as such or in the form of a physiologically acceptable derivative, such as a salt and/or by any source comprising LNnT.

As it is evident to the person skilled in the art, different ingredients may provide different amounts of LNnT in the paediatric supplement or human milk fortifier composition according to the present invention, depending on the nature and amount of the ingredient used. It will be nonetheless routine work to the skilled person to calculate the amount of ingredient needed to provide the claimed amount of LNnT, based on the specification of the specific ingredient provided by the supplier.

In one embodiment, the paediatric supplement or human milk fortifier composition of the present invention comprises LNnT in an amount of at least 30 mg/day. In another embodiment, the paediatric supplement or human milk fortifier composition of the present invention comprises LNnT in an amount ranging from 30 to 50 mg/day.

In one embodiment, the paediatric supplement or human milk fortifier composition of the present invention comprises LNnT in an amount of at least 30 mg. In another embodiment, the paediatric supplement or human milk fortifier composition of the present invention comprises LNnT in an amount ranging from 30 to 50 mg.

In such embodiment, the paediatric supplement or human milk fortifier composition of the present invention delivers in one serving the daily amount of LNnT considered necessary to fill the gap of LNnT between the human breast milk of a mother who delivered vaginally and that of a mother who delivered by C-section.

In one embodiment, the paediatric supplement or human milk fortifier composition according to the present invention may be administered in 1, 2, 3 or 4 daily servings to provide the total daily amounts of LNnT as above described. In such embodiment, as it will be apparent to a person skilled in the art, the amount of LNnT contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Vitamin B6

In one embodiment of the present invention, a supplement or human milk fortifier composition is provided which comprises vitamin B6.

Vitamin B6 may be incorporated in the supplement or human milk fortifier composition according to the present invention as such or in the form of a physiologically acceptable salt and/or by any source comprising vitamin B6. For example, ingredients may be selected in the group consisting of pyridoxine (in the form of pyridoxine hydrochloride [HCl]) and pyridoxal 5′ phosphate (PLP).

As it is evident to the person skilled in the art, different ingredients may provide different amounts of vitamin B6 in the paediatric supplement or human milk fortifier composition according to the present invention, depending on the nature and amount of the ingredient used. It will be nonetheless routine work to the skilled person to calculate the amount of ingredient needed to provide the claimed amount of vitamin B6, based on the specification of the specific ingredient provided by the supplier.

In one embodiment, the paediatric supplement or human milk fortifier composition comprises vitamin B6 is an amount of at least 15 μg/day. In a still further embodiment, the paediatric supplement or human milk fortifier composition comprises vitamin B6 in an amount ranging from 15 to 68 μg/day.

In one embodiment, the paediatric supplement or human milk fortifier composition comprises vitamin B6 is an amount of at least 15 μg. In a still further embodiment, the composition paediatric supplement or human milk fortifier comprises vitamin B6 in an amount ranging from 15 to 68 μg.

In such embodiment, the paediatric supplement or human milk fortifier composition of the present invention delivers in one serving the daily amount of vitamin B6 considered necessary to fill the gap of vitamin B6 between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of vitamin B6 as above described. In such embodiment, as it will be apparent to a person skilled in the art, the amount of vitamin B6 contained in each serving or dosage unit of the composition paediatric supplement or human milk fortifier according to the present invention will be divided by 1, 2, 3 or 4 respectively.

In one embodiment, the composition paediatric supplement or human milk fortifier according to the present invention is intended for consumption once or twice per day.

### Vitamin B2 (Riboflavin)

In one embodiment of the present invention, a supplement or human milk fortifier composition is provided which comprises vitamin B2.

Vitamin B2 may be incorporated in the supplement or human milk fortifier composition of the invention as such or in the form of a physiologically acceptable salt and/or by any source comprising Vitamin B2. For example, ingredients may be selected in the group consisting of riboflavin and riboflavin 5′-monophosphate.

As it is evident to the person skilled in the art, different ingredients may provide different amounts of vitamin B2 in the composition according to the present invention, depending on the nature and amount of the ingredient used. It will be nonetheless routine work to the skilled person to calculate the amount of ingredient needed to provide the claimed amount of vitamin B2, based on the specification of the specific ingredient provided by the supplier.

In one embodiment, the paediatric supplement or human milk fortifier composition comprises vitamin B2 in an amount of at least 9 μg/day. In a further embodiment, the paediatric supplement or human milk fortifier composition comprises vitamin B2 in an amount ranging from 9 μg/day to 19 μg/day.

In one embodiment, the paediatric supplement or human milk fortifier composition comprises vitamin B2 in an amount of at least 9 μg. In a further embodiment, the paediatric supplement or human milk fortifier composition comprises vitamin B2 in an amount ranging from 9 μg to 19 μg.

In such embodiment, the paediatric supplement or human milk fortifier composition of the present invention delivers in one serving the daily amount of vitamin B2 considered necessary to fill the gap of vitamin B2 between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of vitamin B2 as above described. In such embodiment, as it will be apparent to a person skilled in the art, the amount of vitamin B2 contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Thiamine (Vitamin B1)

In one embodiment of the present invention, a paediatric supplement or human milk fortifier composition is provided which comprises thiamine.

Thiamine may be incorporated in the paediatric supplement or human milk fortifier composition of the invention as such or in the form of a physiologically acceptable salt and/or by any source comprising thiamine. For example, ingredients may be selected in the group consisting of thiamine mononitrate and thiamine hydrochloride.

As it is evident to the person skilled in the art, different ingredients may provide different amounts of thiamine in the paediatric supplement or human milk fortifier composition according to the present invention, depending on the nature and amount of the ingredient used. It will be nonetheless routine work to the skilled person to calculate the amount of ingredient needed to provide the claimed amount of thiamine, based on the specification of the specific ingredient provided by the supplier.

In one embodiment, the paediatric supplement or human milk fortifier composition comprises thiamine in an amount of at least 6 μg/day, preferably of at least 6.6 μg/day. In a further embodiment, the paediatric supplement or human milk fortifier composition comprises thiamine in an amount ranging from 6 μg/day to 10 μg/day, preferably in an amount ranging from 6.6 μg/day to 10 μg/day.

In one embodiment, the paediatric supplement or human milk fortifier composition comprises thiamine in an amount of at least 6 μg, preferably in an amount of at least 6.6 μg. In a further embodiment, the paediatric supplement or human milk fortifier composition comprises thiamine in an amount ranging from 6 μg to 10 μg, preferably in an amount ranging from 6.6 μg to 10 μg.

In such embodiment, the paediatric supplement or human milk fortifier composition of the present invention delivers in one serving the daily amount of thiamine considered necessary to fill the gap of thiamine between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of thiamine as above described. In such embodiment, as it will be apparent to a person skilled in the art, the amount of thiamine contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Thiamine Monophosphate

Thiamine monophosphate may be incorporated in the paediatric supplement or human milk fortifier composition of the invention as such or in the form of a physiologically acceptable salt and/or by any source comprising thiamine monophosphate. For example, ingredients may be selected in the group consisting of thiamine mononitrate and thiamine hydrochloride.

As it is evident to the person skilled in the art, different ingredients may provide different amounts of thiamine monophosphate in the paediatric supplement or human milk fortifier composition according to the present invention, depending on the nature and amount of the ingredient used. It will be nonetheless routine work to the skilled person to calculate the amount of ingredient needed to provide the claimed amount of thiamine monophosphate, based on the specification of the specific ingredient provided by the supplier.

In one embodiment, the paediatric supplement or human milk fortifier composition comprises thiamine monophosphate in an amount of at least 10 μg/day. In a further embodiment, the paediatric supplement or human milk fortifier composition comprises thiamine monophosphate in an amount ranging from 10 μg/day to 15 μg/day.

In one embodiment, the paediatric supplement or human milk fortifier composition comprises thiamine monophosphate in an amount of at least 10 μg. In a further embodiment, the paediatric supplement or human milk fortifier composition comprises thiamine monophosphate in an amount ranging from 10 μg to 15 μg.

In such embodiment, the paediatric supplement or human milk fortifier composition of the present invention delivers in one serving the daily amount of thiamine monophosphate considered necessary to fill the gap of thiamine monophosphate between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of thiamine monophosphate as above described. In such embodiment, as it will be apparent to a person skilled in the art, the amount of thiamine monophosphate contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Phosphorus

In one embodiment of the present invention, a paediatric supplement or human milk fortifier composition is provided which comprises phosphorus.

Phosphorus may be incorporated in the paediatric supplement or human milk fortifier of the invention in the form of a physiologically acceptable salt and/or by any source comprising phosphorus. For example, phosphorous may be comprised in the form of sodium phosphate.

As it is evident to the person skilled in the art, different ingredients may provide different amounts of phosphorous in the paediatric supplement or human milk fortifier composition according to the present invention, depending on the nature and amount of the ingredient used. It will be nonetheless routine work to the skilled person to calculate the amount of ingredient needed to provide the claimed amount of phosphorous, based on the specification of the specific ingredient provided by the supplier.

In one embodiment, the paediatric supplement or human milk fortifier composition of the present invention comprises phosphorus in an amount of at least 4.5 mg/day. In another embodiment, the paediatric supplement or human milk fortifier composition of the present invention comprises phosphorus in an amount ranging from 4.5 to 14 mg/day.

In one embodiment, the paediatric supplement or human milk fortifier composition of the present invention comprises phosphorus in an amount of at least 4.5 mg. In another embodiment, the paediatric supplement or human milk fortifier composition of the present invention comprises phosphorus in an amount ranging from 4.5 to 14 mg.

In such embodiment, the paediatric supplement or human milk fortifier composition of the present invention delivers in one serving the daily amount of phosphorous considered necessary to fill the gap of phosphorous between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of phosphorous as above described. In such embodiment, as it will be apparent to a person skilled in the art, the amount of phosphorous contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Calcium

In one embodiment of the present invention, a paediatric supplement or human milk fortifier composition is provided which comprises thiamine.

Calcium may be incorporated in the paediatric supplement or human milk fortifier of the invention in the form of a physiologically acceptable salt and/or by any source comprising calcium. For example, ingredients may be selected in the group consisting of calcium citrate, calcium carbonate or a mixture thereof.

As it is evident to the person skilled in the art, different ingredients may provide different amounts of calcium in the paediatric supplement or human milk fortifier composition according to the present invention, depending on the nature and amount of the ingredient used. It will be nonetheless routine work to the skilled person to calculate the amount of ingredient needed to provide the claimed amount of calcium, based on the specification of the specific ingredient provided by the supplier.

In one embodiment, the paediatric supplement or human milk fortifier composition comprises calcium in an amount of at least 10 mg/day. In a further embodiment, the composition comprises calcium in an amount ranging from 10 to 16 mg/day.

In one embodiment, the paediatric supplement or human milk fortifier composition comprises calcium in an amount of at least 10 mg. In a further embodiment, the paediatric supplement or human milk fortifier composition comprises calcium in an amount ranging from 10 to 16 mg.

In such embodiment, the paediatric supplement or human milk fortifier composition of the present invention delivers in one serving the daily amount of calcium considered necessary to fill the gap of calcium between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of calcium as above described. In such embodiment, as it will be apparent to a person skilled in the art, the amount of calcium contained in each serving or dosage unit of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Vitamin A

In one embodiment of the present invention, a paediatric supplement or human milk fortifier is provided which comprises vitamin A.

Vitamin A may be incorporated in the paediatric supplement or human milk fortifier composition of the invention as such or in the form of a physiologically acceptable derivative such as a salt and/or by any source comprising vitamin A. For example ingredients may be selected in the group consisting of: carrots, red sweet peppers, turnips, orange juice, oranges, tomatoes, dark green leafy vegetables (such as spinach, broccoli and kale), cantaloupe, oranges, tomatoes, apricot, plantains, mangos, passion fruits, squash, yellow corn, soybeans, pistachio nuts, egg yolk, butter, milk, liver, cod liver oil and mixtures thereof. Vitamin pre-mix for fortification purpose is also included.

In one embodiment, vitamin A is provided as such to the paediatric supplement or human milk fortifier composition.

As it is evident to the person skilled in the art, different ingredients may provide different amounts of vitamin A in the paediatric supplement or human milk fortifier composition according to the present invention, depending on the nature and amount of the ingredient used. It will be nonetheless routine work to the skilled person to calculate the amount of ingredient needed to provide the claimed amount of vitamin A, based on the specification of the specific ingredient provided by the supplier.

In one embodiment, the paediatric supplement or human milk fortifier composition comprises vitamin A in an amount of at least 224 μg/day. In a further embodiment, the paediatric supplement or human milk fortifier composition comprises vitamin A in an amount ranging from 224 μg/day to 342 μg/day.

In one embodiment, the paediatric supplement or human milk fortifier composition comprises vitamin A in an amount of at least 224 μg. In a further embodiment, the paediatric supplement or human milk fortifier composition comprises vitamin A in an amount ranging from 224 μg to 342 μg.

In such embodiment, the paediatric supplement or human milk fortifier composition of the present invention delivers in one serving the daily amount of vitamin A considered necessary to fill the gap of vitamin A between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of vitamin A as above described. In such embodiment, as it will be apparent to a person skilled in the art, the amount of vitamin A contained in each serving of the paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Vitamin B9 (Folate or Folic Acid)

In one embodiment of the present invention, a paediatric supplement or human milk fortifier is provided which comprises vitamin B9.

Vitamin B9 may be incorporated in the paediatric supplement or human milk fortifier composition of the invention as folic acid or in the form of a physiologically acceptable derivative, such as a salt and/or by any source comprising vitamin B9 or any mixture thereof, such as leafy green vegetables, (such as spinach, broccoli, and lettuce), beans, peas, and lentils, fruits (such as lemons, bananas, and melon). In one embodiment, vitamin B9 is provided as such to the composition.

As it is evident to the person skilled in the art, different ingredients may provide different amounts of vitamin B9 in the paediatric supplement or human milk fortifier composition according to the present invention, depending on the nature and amount of the ingredient used. It will be nonetheless routine work to the skilled person to calculate the amount of ingredient needed to provide the claimed amount of vitamin B9, based on the specification of the specific ingredient provided by the supplier.

In one embodiment, the paediatric supplement or human milk fortifier composition comprises vitamin B9 in an amount of at least 1.1 μg/day. In a further embodiment, the paediatric supplement or human milk fortifier composition comprises vitamin B9 in an amount ranging from 1.1 μg/day to 9 μg/day.

In one embodiment, the paediatric supplement or human milk fortifier composition comprises vitamin B9 in an amount of at least 1.1 μg. In a further embodiment, the paediatric supplement or human milk fortifier composition comprises vitamin B9 in an amount ranging from 1.1 μg to 9 μg.

In such embodiment, the paediatric supplement or human milk fortifier composition of the present invention delivers in one serving the daily amount of vitamin B9 considered necessary to fill the gap of vitamin B9 between the human breast milk of a mother who delivered vaginally and that of a mother who delivered via C-section.

In one embodiment, the paediatric supplement or human milk fortifier composition according to the present invention may be administered in 1, 2, 3 or 4 daily servings to provide the total daily amounts of vitamin B9 as above described. In such embodiment, as it will be apparent to a person skilled in the art, the amount of vitamin B9 contained in each serving or dosage unit of paediatric supplement or human milk fortifier composition according to the present invention will be divided by 1, 2, 3 or 4 respectively.

In one embodiment, the paediatric supplement or human milk fortifier composition according to the present invention is intended for consumption once or twice per day.

### Method

In one aspect of the present invention, there is provided a method of mitigating nutrient inadequacy in an infant born via C-section, said method comprising:
i) identifying the gap in certain nutrients between the HM composition from mothers who have delivered via C-section and the HM composition from mothers who have delivered vaginally;
ii) providing a paediatric supplement or human milk fortifier according to the invention to mitigate the identified nutritional inadequacies in a child born via C-section.

The identification of the gap in certain nutrients, resulting in nutrient inadequacy, results from the comparative analysis between the HM composition of mothers who delivered vaginally versus the HM composition women who delivered via C-section, using suitable statistical methods known by the skilled person. The analysis of the HM composition may be performed at any period during the breast-feeding period.

The paediatric supplement or human milk fortifier may be provided at any period during the breast-feeding period to an infant born via C-section.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

There now follows a series of non-limiting examples that serve to illustrate the invention.

### EXAMPLES

### Example 1

Four cohorts were evaluated to study differences between the HM composition of mothers who delivered vaginally versus those who delivered via C-section. A brief overview and description of these cohorts are provided in Table II, followed by a more description of the protocol and analysis performed for each of the studies.

TABLE II

| | Study 1 | Study 2 | | Study 4 |
|---|---|---|---|---|
| | Europe | Singapore | | Preemie |
| | NCT01894893 | NCT01805011 | Study 3 | NCT02052245 |
| Study | Longitudinal | Longitudinal | Cross | Longitudinal |
| design | cohort study | cohort study | sectional | cohort study |
| | | | cohort study | |
| Duration of | 4 months | 4 months | 8 months | 4 months |
| follow up | postpartum | postpartum | postpartum | postpartum |
| Population | European | Singaporean | Chinese | Swiss |
| Sample size | C-section: | C-section: | C-section: | C-section: |
| used for | 80 | 19 | 0-4 days 65 | 8 |
| the above | Vaginal: | Vaginal: | 5-11 days 46 | Vaginal: |
| analyses | 237 | 31 | 12-30 days | 20 |
| (with a | | | 43 | |
| split | | | 1-2 months | |
| between | | | 57 | |
| the 2 | | | 2-4 months | |
| groups) | | | 35 | |
| | | | 4-8 months | |
| | | | 34 | |
| | | | Vaginal: | |
| | | | 0-4 days 45 | |
| | | | 5-11 days 50 | |
| | | | 12-30 days | |
| | | | 47 | |
| | | | 1-2 months | |
| | | | 41 | |
| | | | 2-4 months | |
| | | | 55 | |
| | | | 4-8 months | |
| | | | 55 | |

### Study 1(NCT01894893)

### Study Population

Study 1 is a multicentre, longitudinal, observational, exploratory cohort study designed to characterize HM and its association with maternal, infant and child parameters. HM as well as multiple maternal and infant parameters were collected postpartum at 6 visits (V) (V1, 0-3 days; V2, 17±3 days; V3, 30±3 days; V4, 60±5 days; V5, 90±5 days; V6, 120±5 days). Enrolment was performed at multiple sites in 7 European countries, including Spain, France, Italy, Norway, Portugal, Romania, and Sweden. The total duration of participation was 4 months after infant birth. Trained and certified research nurses and assistants collected all data. All data captured were directly entered into a secured web-based database (Medidata Rave edc 5.6.4). The procedures followed were in accordance with the ethical standards of the respective local ethical committees in each country.

### Analysis Population

A total of 370 participants from 7 European countries including Spain (I center), France (3 centers), Italy (I center), Norway (I center), Portugal (3 centers), Romania (2 centers), and Sweden (2 centers) were enrolled for this study and included in the dataset. Participants were counted as pairs of mothers and infants. The analysis was performed on 317 participants (pairs) after removal of pairs who did not satisfy the inclusion-exclusion criteria, pairs with twins and with incomplete information on HM composition

### Statistical Methods

A total of 62 components were considered for this analysis. Values below the level of quantification for a given parameter were replaced by half of the corresponding level of quantification.

To compare levels of the milk components between delivery modes at each visit, two-sided Mann-Whitney U tests were performed. Additionally, to account for confounders, a targeted approach (mixed model) was fitted to each of these 62 components. Such a model was fitted on the log-transformed data to achieve approximate normality of the residuals. Individuals (pairs of a mother and her child) were considered as random effects and the following covariates were considered as fixed effects in the mixed model: visit, delivery mode and the interaction between the two latter. In addition, mother's country, infant's or child's weight at V0 (child's birth), parity, and gestational age were considered as potential confounders and were also added as fixed effects. Due to the log transformation, group differences between delivery modes were computed per visit and were expressed as ratio of geometric means between the vaginal group and the C-section group. Hence, a model-based estimate higher than 1 means that the estimation (geometric mean) is higher in the vaginal group than in C-section group, while an estimate below 1 means that levels of the corresponding parameter are higher in the C-section group. P<0.05 was considered statistically significant.

### NTF (Non-Negative Tensor Factorization)

Non-NTF is a non-supervised approach dedicated to the analysis of longitudinal datasets [25]. Specifically, NTF estimates several factors along the three dimensions of the data space: Participant×Visit×HM component. Each NTF factor represents a particular trend (e.g. increase/decrease). Participant and parameter loadings along each NTF factor reflect the level of similarity for a given participant and parameter with one trend or another. NTF factor loadings are subsequently used to build bi-clusters of participants and milk parameters sharing similar trends. To guide the NTF analysis for greater focus on mode of delivery, pre-selected nutrients were included. Repeated ANOVA with the delivery mode as a main effect on 62 milk nutrients (log-transformed), using JMP version 14.2 (SAS Institute) was conducted to pre-select those nutrients. Participants with missing time points or data were excluded in the repeated ANOVA. In this way, only nutrients were analyzed by NTF that showed some degree of association with the mode of delivery.

### Study 2 (NCT01805011)

### Study Population

Study 2 is an open, single-centre, 1 group study on Singaporean healthy lactating mothers. Milk samples (and other infant parameters) were collected at 3 postpartum visits: 1 month (V1), 2 months (V2) and 4 months (V3) after delivery.

### Analysis Population

The analysis was performed on all 50 enrolled mothers (and their child, no twins).

### Statistical Methods

A total of 60 human milk components were considered for this analysis. Values below the level of quantification for a given parameter were replaced by half of the corresponding level of quantification.

Levels of milk components were compared between delivery modes at each visit by a (two-sided) Wilcoxon rank-sum test. The resulting p-value was reported alongside an estimate of the difference in location between the two groups and a 95% confidence interval of this difference (see the documentation of wilxocon.test function from R package stats).

Comparison of maternal and infant characteristics between delivery modes was performed as follows: for continuous variables a (two-sided) Wilcoxon rank sum test was used to test the null hypothesis that the distributions of C-section and Vaginal have the same location (which amounts to compare their medians if their distributions are symmetric). For categorical variables a Pearson's chi-squared test is used to test the null hypothesis that delivery mode is independent from the current categorical variable (i.e. if the proportions in mode of delivery are independent from the proportions of the categorical variable). If a categorical variable has only one level (with non-missing value), then no test was performed. Given the exploratory nature of the study no multiplicity adjustment was performed and P<0.05 was considered statistically significant.

### Study 3

### Study Population

This is an observational, cross sectional, multi-centre study aiming at evaluating nutrients composition of breast milk and nutrition intake of Chinese lactating mothers in 3 cities of China (Beijing, Suzhou and Guangzhou). In Beijing, Suzhou and Guangzhou there were 220, 180 and 180 of healthy lactating mothers respectively, enrolled at different lactation stages (within 0-4 days, 5-11 days, 12-30 days, 31-60 days, 61-120 days and 121-240 days postpartum). In total, 580 mothers were enrolled.

### Analysis Population

580 mothers were enrolled in the study. The analysis was performed on a subset of 573 mothers (and their child, no twins) who delivered via C-section or by natural birth. Among the 7 mothers that were not included in this analysis: for 3 of them the delivery method is not specified and 4 of them delivered with dystocia of normal labour.

### Statistical Methods

The same methodology than in Study 2 was employed except that here comparisons between delivery modes are performed within each lactating stage on 133 human milk components.

### Study 4(NCT02052245)

### Study Population

Open, single-center, exploratory study on healthy lactating mothers delivering term (gestational age (hereinafter “ga”) between 37 and not above 42 weeks) and preterm (ga between 28 and 32 weeks) infants. A total of 61 mothers (34 in the term group and 25 in the preterm group, 6 mothers in the latter group delivered twins) were enrolled in the study. For subjects who delivered pre-term babies, milk samples were collected once a week until discharge from hospital and then every 2 weeks until 8 weeks after birth (at most 12 visits post-screening). For subjects who delivered term babies, milk samples were collected once a week until 8 weeks after birth (8 visits post-screening).

### Analysis Population

This analysis focused on mothers delivering term infants. Hence, out of the 61 enrolled mothers, the 34 mothers from the term group were considered in this analysis. Following the study protocol, from this population we further removed 6 mothers who dropped out of the study and hence ended up with 28 mothers for the analysis, all of which have delivered unique infants (no multiple births).

### Statistical Methods

The same methodology than in Study 2 was employed. Here, 120 human milk components were considered for analysis.

Table III reports the results of such analysis, showing the gap in certain nutrients between the HM composition from mothers who have delivered via C-section and the HM composition from mothers who have delivered vaginally. Table III also reports the recommended daily intake for each nutrient which would be necessary to close the gap for such nutrients' content in human breast milk received babies born via C-section versus human breast milk received by infants born vaginally, considering that global average human milk consumption is 780 ml/day.

TABLE III

| | | Observed | Median | | | |
|---|---|---|---|---|---|---|
| | | in how | concentrations | | | Daily |
| | | many cohorts | in HM | | | amount to |
| | Direction | and at what | | C- | p- | be |
| Nutrients | of effect | time points | Vaginal | section | value | supplemented |

Fatty acids

| | | | | | | |
|---|---|---|---|---|---|---|
| ALA | Lower in C | Study 1 v1 | 13.6 | 10.6 | ≤0.01 | 23.4 mg/d |
| (18:3 n-3) | section HM | (2 days) | (mg/100 | (mg/100 | | |
| | | | ml) | ml) | | |
| ALA | Lower in C | Study 1 v2 | 21.1 | 15.7 | ≤0.01 | 41.34 mg/d |
| (18:3 n-3) | section HM | (17 days) | (mg/ | (mg/ | | |
| | | | 100 ml) | 100 ml) | | |
| ALA | Lower in C | Study 1 v3 | 22.7 | 15.5 | ≤0.01 | 56.16 mg/d |
| (18:3 n-3) | section HM | (30 days) | (mg/100 | (mg/100 | | |
| | | | ml) | ml) | | |
| ALA | Lower in C | Study 1 v5 | 23.1 | 15.1 | ≤0.01 | 62.4 mg/d |
| (18:3 n-3) | section HM | (90 days) | (mg/100 | (mg/100 | | |
| | | | ml) | ml) | | |
| ALA | Lower in C | Study 1 v6 | 23.6 | 15.1 | ≤0.01 | 66.3 mg/d |
| (18:3 n-3) | section HM | (120 days) | (mg/100 | (mg/100 | | |
| | | | ml) | ml) | | |
| DHA | Lower in C | Study 1 v1 | 11 | 8.3 | 0.01 < | 21.06 mg/d |
| | | (2 days) | (mg/100 | (mg/100 | p ≤ | |
| (22:6 n-3) | section HM | | ml) | ml) | 0.05 | |
| DHA | Lower in C | Study 1 v3 | 11.2 | 9.1 | 0.01 < | 16.38 mg/d |
| (22:6 n-3) | section HM | (30 days) | (mg/100 | (mg/100 | p ≤ | |
| | | | ml) | ml) | 0.05 | |
| EPA | Lower in C | Study 1 V3 | 2.02 | 1.0 | 0.01 < | 7.8 mg/d |
| (20:5n-3) | section | (30 days) | (mg/100 | (mg/ | p ≤ | |
| | | | ml) | 100 ml) | 0.05 | |

HMOs

| | | | | | | |
|---|---|---|---|---|---|---|
| 2′-FL | Lower in C | Study 1 V1 | 3719 | 3267 | <0.01 | 352 mg/d |
| | section HM | (2 days) | (mg/l) | (mg/l) | | |
| | | Study 4 V1 | 3951 | 2818 | 0.05 | 883 mg/d |
| | | (week 1) | (mg/l) | (mg/l) | | |
| LNnT | Lower in C | Study 2 V1 | 261 | 197 | 0.02 | 49.8 mg/d |
| | section HM | (30 days) | (mg/l) | (mg/l) | | |

Gangliosides

| | | | | | | |
|---|---|---|---|---|---|---|
| GD ₃ | Lower in C | Study 1 v1 | 7.7 | 6.4 | 0.01 < | 10.14 mg/d |
| | section HM | (2 days) | (mg/ml) | (mg/ml) | p ≤ | |
| | | | | | 0.05 | |
| GM ₃ | Lower in C | Study 3 v1 | 0.73 | 0.58 | 0.01 | 1.199 mg/d |
| | section HM | (0 to 4 days) | (mg/ml) | (mg/ml) | | |

Micronutrients

| | | | | | | |
|---|---|---|---|---|---|---|
| Vitamin A | Lower in C | Study 3 | 148 | 764 | 0.02 | 280.8 □g/d |
| | section HM | (0-4 days) | (μg/ | (μg/ | | |
| | | | 100 m) | 100 m) | | |
| | | Study 4 V6 | 90 | 53 | 0.04 | 285 □g/d |
| | | (week 6) | (μg/100 | (μg/100 | | |
| | | | ml) | ml) | | |
| Thiamine | Lower in C | Study 4 V3 | 1.32 | 0.25 | 0.03 | 8.34 □g/d |
| | section HM | (week 3) | (μg/100 | (μg/100 | | |
| | | | ml) | ml) | | |
| Thiamine | Lower in C | Study 4 V2 | 6.40 | 4.83 | 0.04 | 12.2 □g/d |
| monophosphate | section HM | (week 2) | (μg/100 | (μg/100 | | |
| | | | ml) | ml) | | |
| Vitamin B2 | Lower in C | Study 4 V4 | 3.82 | 2.40 | 0.04 | 11.00 □g/d |
| (Riboflavin) | section HM | (week 4) | (μg/100 | (μg/100 | | |
| | | | ml) | ml) | | |
| | Lower in C | Study 4 V5 | 3.31 | 1.24 | 0.00 | 16.1 □g/d |
| | section HM | (week 5) | (μg/100 | (μg/100 | | |
| | | | ml) | ml) | | |
| Vitamin B6 | Lower in C | Study 4 V2 | 4.20 | 1.83 | 0.04 | 18.4 □g/d |
| | section HM | (week 2) | (μg/100 | (μg/100 | | |
| | | | ml) | ml) | | |
| | Lower in C | Study 4 V5 | 12.86 | 5.52 | 0.02 | 57.2 □g/d |
| | section HM | (week 5) | (μg/100 | (μg/100 | | |
| | | | ml) | ml) | | |
| Vitamin B9 | Lower in C | Study 4 V3 | 1.07 | 0.10 | 0.02 | 7.56 □g/d |
| | section HM | (week 3) | (μg/100 | (μg/100 | | |
| | | | ml) | ml) | | |
| | Lower in C | Study 4 V4 | 0.33 | 0.10 | 0.03 | 1.79 □g/d |
| | section HM | (week 4) | (μg/100 | (μg/100 | | |
| | | | ml) | ml) | | |
| | Lower in C | Study 4 V5 | 0.28 | 0.10 | 0.04 | 1.40 □g/d |
| | section HM | (week 5) | (μg/100 | (μg/100 | | |
| | | | ml) | ml) | | |
| | Lower in C | Study 4 V8 | 0.70 | 0.10 | 0.02 | 4.68 □g/d |
| | section HM | (week 8) | (μg/100 | (μg/100 | | |
| | | | ml) | ml) | | |
| Calcium | Lower in C | Study 1 V4 | 302 | 286 | <0.05 | 12.9 mg/d |
| | section HM | (60 days) | (mg/l) | (mg/l) | | |
| | | Study 1 V5 | 294 | 279 | <0.05 | 12.0 mg/d |
| | | (90 days) | (mg/l) | (mg/l) | | |
| | | Study 1 V6 | 282 | 267 | <0.01 | 11.7 mg/d |
| | | (120 days) | (mg/l) | (mg/l) | | |
| Phosphorus | Lower in C | Study 1 V1 | 127 | 113 | <0.05 | 11.1 mg/d |
| | section HM | (2 days) | (mg/l) | (mg/l) | | |
| | | Study 1 V1 | 132 | 125 | <0.05 | 5.4 mg/d |
| | | (2 days) | (mg/l) | (mg/l) | | |

### Example 2

Table IV sets out a human milk fortifier composition in accordance with the invention. Said human milk fortifier may be for use to supplement the breast milk produced for a child at least 6 months of age by a mother who has given birth via C-section. Said human milk fortifier is presented as a single daily stick pack dose to be dissolute for example in water or human breast milk.

TABLE IV

| Nutrients | Content per stick pack | |
|---|---|---|
| ALA | 50 | mg |
| (18:3 n-3) | | |
| DHA | 20 | mg |
| (22:6 n-3) | | |
| EPA | 7 | mg |
| (20:5n-3) | | |
| 2′-FL | 500 | mg |
| LNnT | 40 | mg |
| GD₃ | 10 | mg |
| GM₃ | 1 | mg |
| Vitamin A | 250 | μg |
| Thiamine | 8 | μg |
| Thiamine | 12 | μg |
| monophosphate | | |
| Vitamin B2 | 15 | μg |
| (Riboflavin) | | |
| Vitamin B6 | 40 | μg |
| Vitamin B9 | 5 | μg |
| Calcium | 12 | mg |
| Phosphorus | 10 | mg |

### Example 3

Table V sets out a human milk fortifier composition in accordance with the invention. Said human milk fortifier may be for use to supplement the breast milk produced for a child of at least 6 months of age by a mother who has given birth via C-section. Said human milk fortifier is presented in table VII as reconstituted product, for example as added to 100 mL expressed breast milk.

TABLE V

| Nutrients | Content |
|---|---|
| ALA | 20 mg/100 mL |
| (18:3 n-3) | |
| DHA | 11 mg/100 mL |
| (22:6 n-3) | |
| EPA | 2 mg/100 mL |
| (20:5n-3) | |
| 2′-FL | 380 mg/100 mL |
| LNnT | 26 mg/100 mL |
| GD₃ | 7 mg/100 mL |
| GM₃ | 0.7 mg/100 mL |
| Vitamin A | 130 μg/100 mL |
| Thiamine | 1.3 μg/100 mL |
| Thiamine | 6.4 μg/100 mL |
| monophosphate | |
| Vitamin B2 | 3.5 μg/100 mL |
| (Riboflavin) | |
| Vitamin B6 | 8 μg/100 mL |
| Vitamin B9 | 0.5 μg/100 mL |
| Calcium | 295 mg/100 mL |
| Phosphorus | 130 mg/100 mL |

The composition according to the present invention may be formulated with many variations without departing from the scope of the invention as defined in the claims.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A paediatric supplement or human milk fortifier composition comprising at least one nutrient selected from the group consisting of:
alpha linolenic acid (ALA) in a daily amount ranging from 19 to 80 mg;
docosahexaenoic acid (DHA) in a daily amount ranging from 13 to 25 mg;
eicosapentanoic acid (EPA) in a daily amount ranging from 6 to 9 mg;
2-fucosyllactose (2′-FL) in a daily amount ranging from 280 to 1050 mg;
Lacto-N-neotetraose (LNnT) in a daily amount ranging from 30 to 50 mg;
vitamin A in a daily amount ranging from 224 to 342 kg;
thiamine in a daily amount ranging from 6.6 to 10 kg;
thiamine monophosphate in a daily amount ranging from 10 to 15 kg;
vitamin B2 in a daily amount ranging from 9 to 19 kg;
vitamin B6 in a daily amount ranging from 15 to 68 kg;
vitamin B9 in a daily amount ranging from 1.1 to 9 kg;
calcium in a daily amount ranging from 10 to 16 mg;
phosphorus in a daily amount ranging from 4.5 to 14 mg;
GD3 in a daily amount ranging from 8 to 12 mg; and
GM3 in a daily amount ranging from 1 to 1.5 mg; and
combinations thereof.

2. (canceled)

3. A paediatric supplement or human milk fortifier composition according to claim 1 wherein the composition comprises:
alpha linolenic acid (ALA) in a daily amount ranging from 19 to 80 mg;
docosahexaenoic acid (DHA) in a daily amount ranging from 13 to 25 mg;
eicosapentanoic acid (EPA) in a daily amount ranging from 6 to 9 mg; and
2-fucosyllactose (2′-FL) in a daily amount ranging from 280 to 1050 mg.

4. A paediatric supplement or human milk fortifier composition according to claim 3 which also comprises at least one nutrient selected from the group consisting of:
Lacto-N-neotetraose (LNnT) in a daily amount ranging from 30 to 50 mg;
vitamin A in a daily amount ranging from 224 to 342 kg;
thiamin in a daily amount ranging from 6.6 to 10 kg;
thiamin monophosphate in a daily amount ranging from 10 to 15 kg;
vitamin B2 in a daily amount ranging from 9 to 19 kg;
vitamin B6 in a daily amount ranging from 15 to 68 kg;
vitamin B9 in a daily amount ranging from 1.1 to 9 kg;
calcium in a daily amount ranging from 10 to 16 kg;
phosphorus in a daily amount ranging from 4.5 to 14 mg;
GD3 in a daily amount ranging from 8 to 12 mg; and
GM3 in a daily amount ranging from 1 to 1.5 mg; and
combinations thereof.

5. A paediatric supplement or human milk fortifier composition according to claim 1 which comprises at least one nutrient selected from the group consisting of
calcium in a daily amount ranging from 10 to 16 mg;
phosphorus in a daily amount ranging from 4.5 to 14 mg;
GD3 in a daily amount ranging from 8 to 12 mg; and
GM3 in a daily amount ranging from 1 to 1.5 mg;
and
Lacto-N-neotetraose (LNnT) in a daily amount ranging from 30 to 50 mg.

6-8. (canceled)

9. A paediatric supplement or human milk fortifier composition according to claim 1, wherein said composition further comprises one or more ingredient selected from the group consisting of additional vitamins, additional minerals, protein, additional carbohydrates, and probiotics.

10. A method for providing adequate nutrition to a child born via C-section comprising administering a paediatric supplement or human milk fortifier composition comprising at least one nutrient selected from the group consisting of:
alpha linolenic acid (ALA) in a daily amount ranging from 19 to 80 mg;
docosahexaenoic acid (DHA) in a daily amount ranging from 13 to 25 mg;
eicosapentanoic acid (EPA) in a daily amount ranging from 6 to 9 mg;
2-fucosyllactose (2′-FL) in a daily amount ranging from 280 to 1050 mg;
Lacto-N-neotetraose (LNnT) in a daily amount ranging from 30 to 50 mg;
vitamin A in a daily amount ranging from 224 to 342 μg;
thiamine in a daily amount ranging from 6.6 to 10 μg;
thiamine monophosphate in a daily amount ranging from 10 to 15 μg;
vitamin B2 in a daily amount ranging from 9 to 19 μg;
vitamin B6 in a daily amount ranging from 15 to 68 μg;
vitamin B9 in a daily amount ranging from 1.1 to 9 μg;
calcium in a daily amount ranging from 10 to 16 mg;
phosphorus in a daily amount ranging from 4.5 to 14 mg;
GD3 in a daily amount ranging from 8 to 12 mg; and
GM3 in a daily amount ranging from 1 to 1.5 mg; and
combinations thereof to the child.

11. A method according to claim 10 for use in preventing sub-optimal growth and development in a child born via C-section.

12. A method according to claim 10 for use in the prevention and or treatment of inflammatory process in the child.

13. A method according to claim 10 for use in optimising the gut microbiota and/or preventing sub-optimal gut microbiota in the child.

14. A method according to claim 10 for use in promoting cognitive development in the child.

15. A method according to claim 10 for use in promoting immunity development in the child.

16-20. (canceled)

21. Method of mitigating nutrient inadequacy in an infant born via C-section, said method comprising:
i) identifying the gap in certain nutrients between the human breast milk composition from mothers who have delivered via C-section and the human breast milk composition from mothers who have delivered vaginally; and
ii) providing a paediatric supplement or human milk fortifier composition comprising at least one nutrient selected from the group consisting of:
alpha linolenic acid (ALA) in a daily amount ranging from 19 to 80 mg;
docosahexaenoic acid (DHA) in a daily amount ranging from 13 to 25 mg;
eicosapentanoic acid (EPA) in a daily amount ranging from 6 to 9 mg;
2-fucosyllactose (2′-FL) in a daily amount ranging from 280 to 1050 mg;
Lacto-N-neotetraose (LNnT) in a daily amount ranging from 30 to 50 mg;
vitamin A in a daily amount ranging from 224 to 342 μg;
thiamine in a daily amount ranging from 6.6 to 10 μg;
thiamine monophosphate in a daily amount ranging from 10 to 15 μg;
vitamin B2 in a daily amount ranging from 9 to 19 μg;
vitamin B6 in a daily amount ranging from 15 to 68 μg;
vitamin B9 in a daily amount ranging from 1.1 to 9 μg;
calcium in a daily amount ranging from 10 to 16 mg;
phosphorus in a daily amount ranging from 4.5 to 14 mg;
GD3 in a daily amount ranging from 8 to 12 mg; and
GM3 in a daily amount ranging from 1 to 1.5 mg; and
combinations thereof to mitigate the identified nutritional inadequacies in a child born via C-section.
